(19) United States
(12) Patent Application Publication
(10) Pub. No.: US 2021/0378802 A1
(43) Pub. Date: Dec. 9, 2021

(54) DENTAL DOWNDRAFT SUCTION

(71) Applicant: Robert Leon Jeanmenne, Port Matilda, PA (US)

(72) Inventor: Robert Leon Jeanmenne, Port Matilda, PA (US)

(21) Appl. No.: 16/893,399

(22) Filed: Jun. 4, 2020

(51) Int. Cl. A61C 17/06; A61C 17/08; A61C 17/10

(52) U.S. Cl. CPC A61C 17/065; A61C 17/10; A61C 17/08

(57) ABSTRACT

An article of manufacture for providing a dental droplet extraction device is disclosed. The device is created around a large collection basin having a funnel-like opening at its top. An exit port is located at the bottom of the large collection basin. The exit port is connected to a high-volume dental line to provide negative pressure that suctions all of the aerosolized droplets created within the patient's mouth while receiving treatment. The removed particles may be trapped in a filter located in the dental suction lines.

### CROSS-REFERENCE TO RELATED APPLICATION

This application is related to U.S. Design patent application Ser. No. __/______, titled “Dental Downdraft Suction,” and that is concurrently filed with the present application and is commonly owned. The entire application is incorporated herein by reference in its entirety.

### TECHNICAL FIELD

This application relates in general to an article of manufacture for providing a personal protection device, and more specifically, to an article of manufacture for providing a dental aerosol reduction and source capture device.

### BACKGROUND

Dental treatments typically generate aerosolized droplets within the patient's mouth during a procedure. These aerosolized droplets may contain unwanted and possibly dangerous pathogens when the patient is ill, whether or not they realize that they are ill. Dentists, their staff members, and other patients may be exposed to these pathogens that may float through the air in a dental office. This exposure to these pathogens may result in additional infections that spread the pathogen through a community. Even the act of a patient breathing will generate microdroplets that can make a potentially infections cloud inside the dental operatory. This cloud can infect the next patient in that room, or the dental personnel.

Therefore, a need exists for a dental droplet reduction device that may remove the aerosolized droplets as they are exiting a patient's mouth to be captured and removed from the local environment. This device should use facilities already known to be in most dental offices while not impeding a dentist from providing treatment within the patient's mouth. The present invention attempts to improve the working environment in dental offices to address the problems identified herein.

### SUMMARY

In accordance with the present invention, the above and other problems are solved by providing an article of manufacture for a dental droplet extraction device creating a downdraft suction or downdraft vent according to the principles and example embodiments disclosed herein.

In one embodiment, the present invention is an article of manufacture for providing a dental droplet extraction device. The device is created around a large collection basin having a funnel-like opening at its top. An exit port is located at the bottom of the large collection basin. The exit port is connected to a dental high volume suction line to provide negative pressure that suctions much of the aerosolized droplets and exhaled microparticles created within the patient's mouth while receiving treatment. The removed particles may be trapped in a filter located in the dental suction unit.

In another embodiment, the present invention is a system for providing a dental droplet extraction from a dental patient. The system has a dental aerosol collection device, a dental suction line coupled to a suction pump for providing negative air pressure within the collection basin; and a filter device located between the dental aerosol collection device and the suction pump to remove aerosol and other particles from about the funnel-like opening of the dental aerosol collection device. The dental aerosol collection device has a funnel-like opening about a top surface and tapered inner surface creating a collection basin and leading to an exit cavity along a bottom surface, an exit port coupled to the exit cavity leading away from the dental aerosol collection device, and a flat plane coupled to a rear surface of the dental aerosol collection device providing support for the collection basin when worn by a dental patient. The flat plane comprises a pair of connection opening for attaching alligator clips coupled to a support line for placement on the dental patient about a neck.

The foregoing has outlined rather broadly the features and technical advantages of the present invention in order that the detailed description of the invention that follows may be better understood. Additional features and advantages of the invention will be described hereinafter that form the subject of the claims of the invention.

It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features that are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only, and is not intended as a definition of the limits of the present invention.

### BRIEF DESCRIPTION OF THE DRAWINGS

Referring now to the drawings in which like reference numbers represent corresponding parts throughout:

FIG. 1 illustrates an example embodiment of a dental droplet extraction device according to the present invention.

FIG. 2 illustrates an upper front view of an example embodiment of a dental droplet extraction device according to the present invention.

FIG. 3 illustrates a lower front view of an example embodiment of a dental droplet extraction device according to the present invention.

FIG. 4 illustrates an offset top view of an example embodiment of a dental droplet extraction device according to the present invention

FIG. 5 illustrates a top perspective view of an example embodiment of a dental droplet extraction device according to the present invention.

FIG. 6 illustrates a bottom view of an example embodiment of a dental droplet extraction device according to the present invention.

FIG. 7 illustrates a top view of an example embodiment of a dental droplet extraction device according to the present invention.

### DETAILED DESCRIPTION

This application relates in general to an article of manufacture for providing a personal protection device, and more specifically, to an article of manufacture for providing a dental droplet extraction device according to the present invention.

Various embodiments of the present invention will be described in detail with reference to the drawings, wherein like reference numerals represent like parts and assemblies throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

In describing embodiments of the present invention, the following terminology will be used. The singular forms “a,” “an,” and “the” include plural referents unless the context clearly dictates otherwise. Thus, for example, reference to “a needle” includes reference to one or more of such needles and “etching” includes one or more of such steps. As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. As used herein, the singular forms “a,” “an,” and “the” are intended to include the plural forms as well, unless the context clearly indicates otherwise.

It further will be understood that the terms “comprises,” “comprising,” “includes,” and “including” specify the presence of stated features, steps or components, but do not preclude the presence or addition of one or more other features, steps or components. It also should be noted that in some alternative implementations, the functions and acts noted may occur out of the order noted in the figures. For example, two figures shown in succession may in fact be executed substantially concurrently or may sometimes be executed in the reverse order, depending upon the functionality and acts involved.

As used herein, the term “about” means that dimensions, sizes, formulations, parameters, shapes, and other quantities and characteristics are not and need not be exact, but may be approximated and/or larger or smaller, as desired, reflecting tolerances, conversion factors, rounding off, measurement error and the like, and other factors known to those of skill. Further, unless otherwise stated, the term “about” shall expressly include “exactly,” consistent with the discussion above regarding ranges and numerical data.

The terms “customer,” and “patient” refer to an entity, e.g. a human, using a dental droplet extraction device associated with the invention. The term user herein refers to one or more users.

The term “invention” or “present invention” refers to the invention being applied for via the patent application with the title “Dental Downdraft Suction.” Invention may be used interchangeably with dental device.

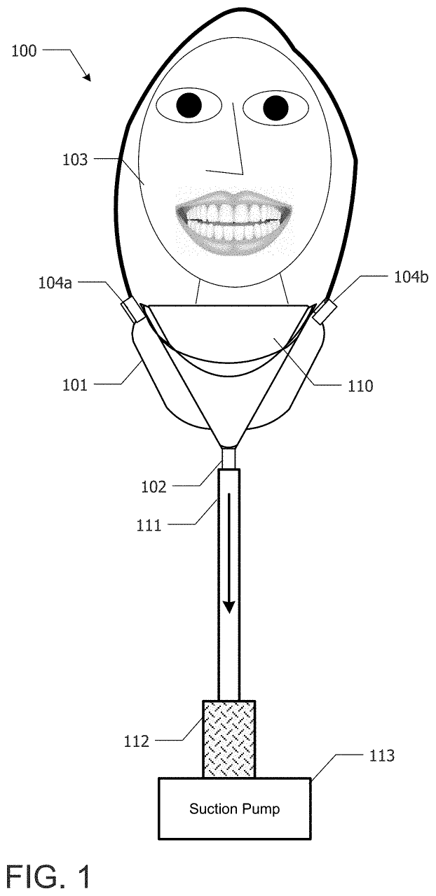

In general, the present disclosure relates to an article of manufacture for providing a personal protection device. To better understand the present invention, FIG. 1 illustrates an example embodiment of an extra oral suction device that acts as a dental droplet extraction device according to the present invention. This device 100 attaches to the end of the high-volume dental suction line 111 found in all dental offices. The device 100 is placed on the dental patient's chest and attached with alligator clips 104a-b using a support line like the dental patient napkin is attached. The device 100 is typically adjusted using the using a variable length cord attached to the alligator clips 104a-b to rest several inches below the patient's chin. The device 100 functions to create a negative area of pressure around the patient's mouth. This will remove harmful microdroplets from the area around the patient's face by suctioning the surrounding air into the dental suction lines 111 already built into the dentist's chair. The dental suction lines 111 are attached to a suction pump 113 that provides the negative pressure. A filter device 112 may also be included between the dental suction line 111 and the suction pump 113 to capture any pathogens contained in the aerosolized droplets. In some embodiments, the filter device material attachment device 112 may be part of the suction pump 113 rather than a separate item as shown in FIG. 1. This device 100 helps to protect the operating personnel and other patients in the office from inhaling aerosolized droplets created while providing treatment.

The device 100 can be constructed of disposable polypropylene, an ideal form, or made of metal or thicker plastic to be reusable/autoclavable (cleanable). The device 100 attaches to the high-volume dental suction line 111 by a standard 11 mm diameter tube at its tip. The top of the device 100 opens up to form a collar-like area 110 several inches below the patient's chin. The device 100 has a flat plane 101 that will stabilize it on the patient's chest while the patient is upright or supine.

A dentist using it would ideally need to have two high-volume suctions lines, one for the device and one for standard suction use in the mouth. These additional suction lines may easily be installed by a technician and is not part of this patent. The device 100 is simply the part that attaches to the suction line, like a vacuum hose attachment.

The device 100 may be used during every procedure to vacuum out the air around the patient's face and mouth so that aerosol, water droplets, breath, viruses such as covid-19, bacteria, and any other contaminants from the patient's mouth will be vacuumed away from the dental operators. This device 100 is to reduce aerosolized microbes from spreading into the air of the dental office.

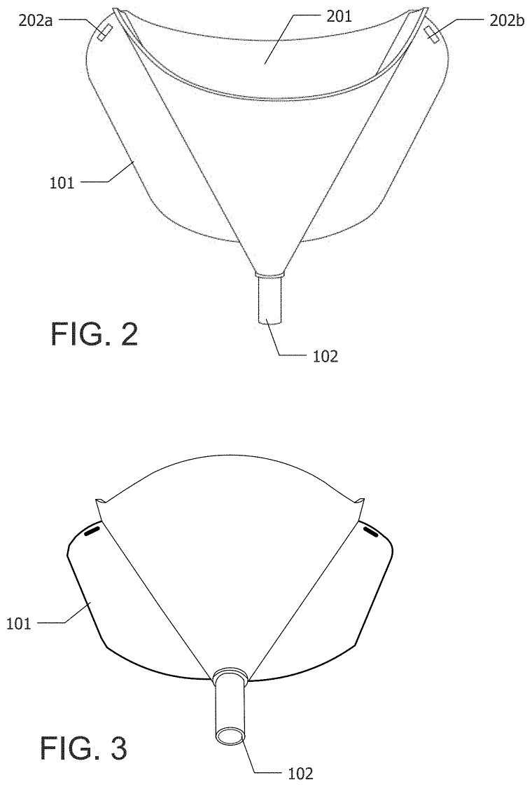

FIG. 2 illustrates an upper front view of an example embodiment of a dental aerosol collection device according to the present invention. In this view of the device 100, a funnel-like opening 201 is seen at the top of the device that tapers downward towards an exit cavity. An exit port 202 is located at the bottom of the device 100 and coupled to the exit cavity for connecting the exit port 202 to the dental suction line 111. Two attachment slots 202a-b are located near the top of the flat plane 101 on the back of the device 100. The attachment slots 202a-b are used to couple the alligator clips 104a-b to the device 100.

FIG. 3 illustrates a lower front view of an example embodiment of a dental aerosol collection device according to the present invention. This view shows the flat plane 101 at the back of the device 100 and the exit port 102 at the bottom of the device. The remaining visible part of the device 100 is the outside surface of the funnel-like opening 102.

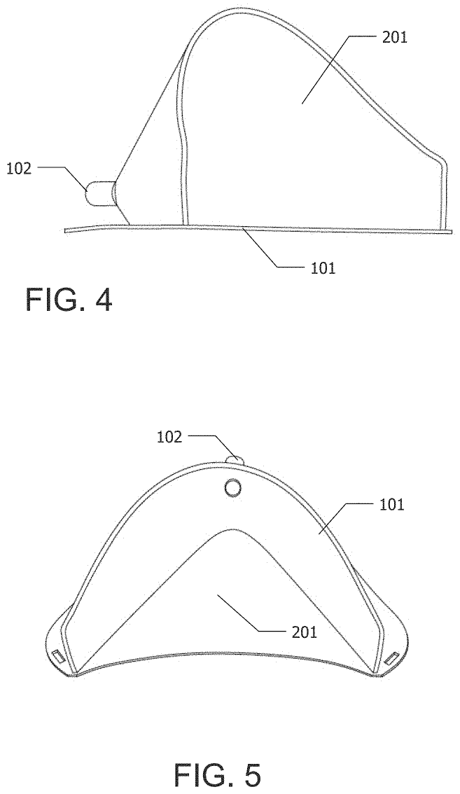

FIG. 4 illustrates an offset top view of an example embodiment of a dental aerosol collection device according to the present invention. In this view, the funnel-like opening 102 is shown as a large collection basin 201 that opens to the top of the device 100. The exit port 102 is visible at the opposite side of the device. The flat plane 101 is supporting the large collection basin 201.

FIG. 5 illustrates a top perspective view of an example embodiment of a dental droplet extraction device according to the present invention. In this view, the large collection basin 201 is shown. A flat back surface of the large collection basin 201 is part of the flat plane 101. The exit port 102 is seen directed into the funnel base of the large collection basin 201.

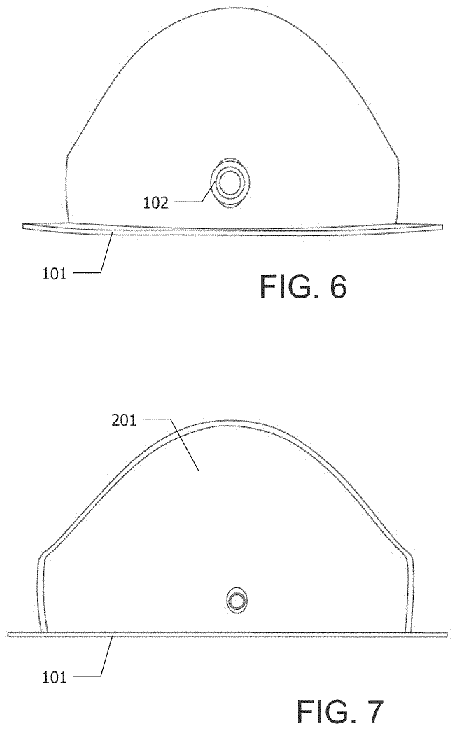

FIG. 6 illustrates a bottom view of an example embodiment of a dental droplet extraction device according to the present invention. In this view, the exit port 102 is seen as the view looks through the port to the large collection basin 201. The device 100 once again is supported on top of the flat plane 101.

FIG. 7 illustrates a top view of an example embodiment of a dental droplet extraction device according to the present invention. In this view, the large collection basin 201 is seen as viewed from the funnel-like opening 102 through to the exit port 102. The flat plane is seen at the bottom of the device 100.

Even though particular combinations of features are recited in the present application, these combinations are not intended to limit the disclosure of the invention. In fact, many of these features may be combined in ways not specifically recited in this application. In other words, any of the features mentioned in this application may be included to this new invention in any combination or combinations to allow the functionality required for the desired operations.

No element, act, or instruction used in the present application should be construed as critical or essential to the invention unless explicitly described as such. Further, the phrase “based on” is intended to mean “based, at least in part, on” unless explicitly stated otherwise. Any singular term used in this present patent application is applicable to its plural form even if the singular form of any term is used.

What is claimed:

1. An article of manufacture for providing a dental droplet extraction device, the article comprises:
a dental aerosol collection device having a funnel-like opening about it top surface and tapered inner surface creating a collection basin and leading to an exit cavity along a bottom surface;
an exit port coupled to the exit cavity leading away from the dental aerosol collection device; and
a flat plane coupled to a rear surface of the dental aerosol collection device providing support for the collection basin when worn by a dental patient.

2. The article of manufacture according to claim 1, wherein the flat plane comprises a pair of connection opening for attaching alligator clips coupled to a support line for placement on the dental patient about a neck.

3. The article of manufacture according to claim 1, wherein the exit port is coupled to a dental suction line and a filter device for providing negative air pressure within the collection basin to remove aerosol and other particles from about the funnel-like opening of the dental aerosol collection device.

4. The article of manufacture according to claim 1, wherein the article is made from a biodegradable material.

5. The article of manufacture according to claim 1, wherein the article is made from a solid reusable material capable of being sterilized.

6. The article of manufacture according to claim 5, wherein the solid reusable material capable of being sterilized is metal.

7. The article of manufacture according to claim 5, wherein the solid reusable material capable of being sterilized is heavy plastic.

8. The article of manufacture according to claim 3, wherein the exit port couples to an 11 millimeter suction line.

9. The article of manufacture according to claim 5, wherein the flat plane includes a collar-like shape to aid in its placement on the dental patient below a neck and mouth.

10. A system for providing a dental droplet extraction from a dental patient, the system comprises:
a dental aerosol collection device, the dental aerosol collection device comprises:
a funnel-like opening about a top surface and tapered inner surface creating a collection basin and leading to an exit cavity along a bottom surface;
an exit port coupled to the exit cavity leading away from the dental aerosol collection device; and
a flat plane coupled to a rear surface of the dental aerosol collection device providing support for the collection basin when worn by a dental patient;
a dental suction line coupled to a suction pump for providing negative air pressure within the collection basin; and
a filter device located between the dental aerosol collection device and the suction pump to remove aerosol and other particles from about the funnel-like opening of the dental aerosol collection device;
wherein the flat plane comprises a pair of connection opening for attaching alligator clips coupled to a support line for placement on the dental patient about a neck.

11. The system according to claim 10, wherein the article is made from a biodegradable material.

12. The system according to claim 10, wherein the article is made from a solid reusable material capable of being sterilized.

13. The system according to claim 12, wherein the solid reusable material capable of being sterilized is metal.

14. The system according to claim 12, wherein the solid reusable material capable of being sterilized is heavy plastic.

15. The system according to claim 10, wherein the exit port couples to an 11 millimeter suction line.
